SEQUENCE LISTING (4 sequences)

SEQ ID NO: 1 - protein, 175 aa, Unknown
  Amino Acid Sequence of Human Alpha B Crystallin
MDIAIHHPWIRRPFFPFHSPSRLFDQFFGEHLLESDLFPTSTSLSPFYLRPPSFLRAPSWFDTGLSEMRLEKDRFSVNLNVKHFSPEELKVKVLGDVIEVHGKHEERQDEHGFISREFHRKYRIPADVDPLAITSSLSSDGVLTVNGPRKQVSGPERTIPITREEKPAVTAAPKK

SEQ ID NO: 2 - protein, 8 aa, Unknown
  Fragment 1 of purified human 23 kDa protein containing alpha B crystallin sequences
IPADVDPL

SEQ ID NO: 3 - protein, 4 aa, Unknown
  Fragment 2 of purified human 23 kDa protein containing alpha B crystallin sequence
YLRP

SEQ ID NO: 4 - protein, 13 aa, Unknown
  Fragment 3 of purified human 23 kDa protein containing alpha B crystallin sequences
APSWFDTGLSEMR

(19) United States
(12) Patent Application Publication
(10) Pub. No.: US 2005/0013824 A1
(43) Pub. Date: Jan. 20, 2005

(54) Alpha B crystallin for use in diagnosis and therapy of auto-immune diseases in particular multiple sclerosis

(76) Inventors: Johannes M. Van Noort, Rijswijk (NL); Arianne C. Van Sechel, Rijswijk (NL)

Correspondence Address: NORRIS, MCLAUGHLIN & MARCUS, P.A., 875 THIRD AVE, 18TH FLOOR, NEW YORK, NY, 10022, US

(21) Appl. No.: 10/732,586

(22) Filed: Dec. 10, 2003

Related U.S. Application Data
(62) Division of application No. 09/668,969, filed on Sep. 25, 2000, now abandoned.

(30) Foreign Application Priority Data
Jun. 9, 1994 (EP) 94201653.6

(51) Int. Cl. A61K 39/00

(52) U.S. Cl. 424/185.1

(57) ABSTRACT

The present invention discloses the first medical use of alpha B crystallin or derivatives thereof in the field of auto-immune diseases, particularly multiple sclerosis. It also discloses diagnostic methods and tools therefor based upon alpha B crystallin for the detection of multiple sclerosis. Human alpha B crystallin is a known protein in itself, known to be present in the eye and in small amounts in the white matter of the nerve system, which is affected by multiple sclerosis. By using a novel and inventive approach to test for ability to induce auto-immunity, alpha B crystallin has been shown by the present invention to be an important auto-antigen. Such methods are also part of the invention.

### BACKGROUND

The invention relates to the field of immunology, in particular to the field of auto-immune diseases. More in particular, it relates to the diagnosis and therapy of autoimmune diseases, especially multiple sclerosis. Auto-immune diseases are diseases wherein the defense mechanism of the body against unwanted materials, such as bacteria, viruses, etc. looses the ability to discriminate between unwanted and self material and attacks parts of the own material, resulting in destruction of essential tissues and functions. To allow for a better understanding of the present invention a short introduction to how this defense mechanism operates is given below.

The Immune System and its Control

The immune system of man and animal is a delicately balanced system designed to monitor and remove pathogens and foreign substances from the living body. Every day, each individual encounters numerous different bacteria, viruses or other substances that may jeopardize normal body functions when residing or even propagating within body tissues.

Immunologists refer to any proteinaceous material foreign to the body itself as ‘antigen’. The immune system comprises a variety of different cells that may respond to antigens from every conceivable source.

Specific responses of the immune system are controlled by a particular type of cells, called helper T cells, that act as monitoring and regulatory cells. Such helper T cells may activate cytotoxic T cells (to destroy affected cells or tissues), B cells (to produce and secrete antibodies) or macrophages (to ingest material and secrete protein-degrading enzymes and damaging oxidative radicals) Thus, helper T cells play a pivotal role in determining when the immune system comes into action and against exactly which structures it will be targeted.

This latter element, the target specificity of the immune response as recognized by helper T cells, is defined by a structural sample of the antigen, viz. protein fragments derived from it. As a rule, helper T cells screen for the presence of antigen by examining sets of protein fragments that are presented to them by so-called accessory cells, frequently macrophages. Such accessory cells ingest antigen and partially digest the material by protein-degrading enzymes, irrespective of the nature or source of the antigen.

Next, resulting protein fragments derived from the antigen are trapped (still within the accessory cell) by special proteins designed to capture and present protein fragments. These proteins, encoded in the major histocompatibility complex (MHC) class II region in the genome, are referred to as class

II MHC proteins. MHC proteins, complexed in a 1:1 ratio with a protein fragment emerging from the degradative processes within the accessory cells, become surface exposed and can reside at the cell surface for days. Thus, they will present to passing helper T cells a structural sample of antigens in the form of the captured protein fragments [1].

The immune system comprises a population of helper T cells, each of which possesses a surface receptor structure with its own individual target specificity [2]. Together, however, the many millions of different helper T cells receptors on the different cells are capable of recognizing virtually every protein fragment. This is a prerequisite for the immune system's potential to recognize an endless variety of structurally different entities. At the same time however, it poses a significant risk that responses may occur also to structures that belong to the body itself. An accurate balance between responding to possibly pathogenic antigen and being tolerant to the body's own tissues is crucial to a healthy immune system [3].

Unfortunately, the balance between aggression and tolerance is not always maintained. Especially tissue-specific structures within the body may become the target of autoreactive immune responses and disease may ensue [4]. In case of aberrant responses to insulin-producing islets of

Langerhans within the pancreas, insulin-dependent diabetes may result. When cartilage structures are attacked, rheumatoid arthritis follows and if something within the brain's white matter triggers the immune system, multiple sclerosis may develop. Although these diseases result in quite different symptoms in the individuals they affect, immunologists consider them to have similar origins: an uncontrolled and perpetuating immune response against one or more antigens within the body's own tissues: autoimmunity.

In the western world, about 3% of all people suffer from autoimmune responses. About one in every thousand suffers from multiple sclerosis (MS). MS is characterized by scattered regions of inflammation (lesions) within white matter of the central nervous system, brain and spinal cord [5]. This white matter is mainly composed of a material specific to central nervous system (CNS) tissue, viz. myelin, that is wrapped around axons in layers of insulating membranes. Nerve signals controlling every imaginable body function travel through axons from one nerve cell to another. Unlike current through electric wire, however, signal conduction in the CNS is achieved by so called saltatory conduction. Electrochemical potentials are not gradually transported along the entire axon length, but jump between small spots of naked axons (nodes of Ranvier) that exist between the patches of myelin.

By jumping from spot to spot, nerve signal transduction in the central nervous system is rapid and efficient. In MS, focal inflammatory responses in the CNS cause the degradation of the myelin layer and their complete removal from axons.

Demyelinated axons become much less efficient in nerve signal conduction since the saltatory mode of transduction is no longer possible. Nerve signals will thus take more time to travel the required distances and they will loose part of their potential along the way. Even if the inflammatory response passes, damage persists since remyelination of naked axons occurs to an only very limited extent. Instead, scar tissue develops. It is for this reason that damage in the CNS as the result of MS slowly accumulates.

It is generally believed that the target antigen for the autoimmune response in MS is to be found in CNS myelin [5, 6].

Myelin consists of differentiated oligodendrocyte plasmamembranes, containing about 75% lipids and about 25% proteins [7]. Since helper T cells only respond to protein fragments, no major role is anticipated for the different lipid components of myelin in triggering autoimmune responses.

The 25% protein mass of myelin however, is highly complex in composition. Several levels of complexity may be discerned.

First, CNS myelin contains a large number of different proteins. Predominant protein components exist, viz. myelin basic protein (MBP) and proteolipid protein (PLP), that represent about 15 and 35% of the total protein mass, respectively. In addition, however, a multitude of other protein components have been found to be specifically associated with CNS myelin. Some of these proteins have been under study as the result of the availability of monoclonal antibodies. These are for example myelin-associated glycoprotein (MAG) and myelin/oligodendrocyte glycoprotein (MOG), which represent only very minor amounts of protein mass in the order of less than 1%. Numerous other proteins including a variety of different enzymes have been found in myelin as well [reviewed in 8].

Secondly, molecular biological studies of myelin proteins have consistently revealed the presence of different isoforms [reviewed in 9]. More as a rule rather than as an exception, primary transcripts of myelin proteins may be spliced differentially into different isoforms. Such molecular species differ from one another in the number of exons they represent.

For example, MBP is currently known to exist in at least six different isoforms in mice.

Thirdly, every myelin protein examined to date contains at least one type of post-translational modification. These modifications include acetylations, acylation, glycosylation, methylation, phosphorylation etc. Finally, both isoform expression and extent and patterns of post-translational modifications may vary in time according to developmental stage or health. In the development of mice, constantly different ratios of different MBP isoforms can be found in the brain. When the animals are struck by infections in the CNS, such patterns may change and different modifications may appear. Without any additional information, no single myelin protein may be considered to be a more relevant antigenic target than any other myelin protein. The mere abundance of e.g. MBP or PLP does not immediately imply these proteins to be any more immunogenic than other myelin components. Numerous examples exist indicating that helper T-cell responses to protein mixtures may well be directed at very minor components [8]. Despite this consideration, much of the effort in immunological research on MS has been devoted to MBP. Without any doubt, the simple fact that MBP is a water soluble and readily available protein for experimental studies has played a major role in its selection as candidate autoantigen.

However, so far there is no evidence that any of the above mentioned proteins represents a major auto-antigen in autoimmune diseases, particularly MS.

The need for identifying such an auto-antigen is apparent from the above, if only to be able to diagnose MS at a much earlier stage and to design rational intervention strategies.

Moreover, knowledge of the auto-antigen can be used to develop specific therapies for MS instead of the aspecific immunemodulation which is used to date. Based on the autoantigen it will be possible to develop vaccines or schemes for inducing tolerance [10-12]. The present invention provides an important candidate for being a major auto-antigen in the development of MS and a target for diagnosis and therapeutic intervention.

### BRIEF DESCRIPTION OF THE DRAWINGS

FIG. 1 is a graph showing reversed-phase HPLC fractionation of total CNS myelin protein;

FIG. 2 is a series of graphs showing proliferative responses of human T cells to fractionated CNS myelin proteins, after a 2 week priming with total myelin protein. The left series of panels illustrates responses to mylelin proteins from control human brain; the rights series to proteins from MS-affected brain. Alpha B crystallin is contained in fraction 8 of the HPLC fraction series.

FIG. 3 is the amino Acid sequence of human alpha B crystallin.

FIG. 4—Proliferative responses to alpha B-crystallin (counts per minute) are shown for T cells derived from either lymph nodes (left hand panels A, C, E and G) or spleen (right hand panels B, D, F and H) of alpha B-crystallin-knock-out mice following immunization with recombinant human alpha B-crystallin. On days 10 (panels A and B) and 20 (panels C and D), a marked antigen-specific response has developed. Oral administration of recombinant human alpha B-crystallin for five days between days 20 and 24 leads to a strong reduction of the response (panels E and F), which is not seen for the control group that are fed only phosphate-buffered saline during the same period (panels G and H).

A Highly Immunodominant CNS Myelin Protein: Alpha B Crystallin

By applying a novel approach to the study of myelin-reactive human helper T cells, a new antigen has now been identified that is likely to serve as crucial autoantigen in inflammatory responses in MS.

In previous studies documented throughout the years, the approach has always been to purify an individual protein from

CNS myelin and subsequently examine its ability to trigger helper T cell responses in man or demyelinating T-cell responses in laboratory animals. Although relevant information on the principles of autoimmune responses could thus be generated, this approach did never address the crucial question whether or not the selected antigen was actually immunodominant to helper T cells in the context of all myelin proteins together. Yet, it is very likely that a challenge of the immune system with all myelin proteins at the same time is close to what happens in MS, at least in ongoing disease.

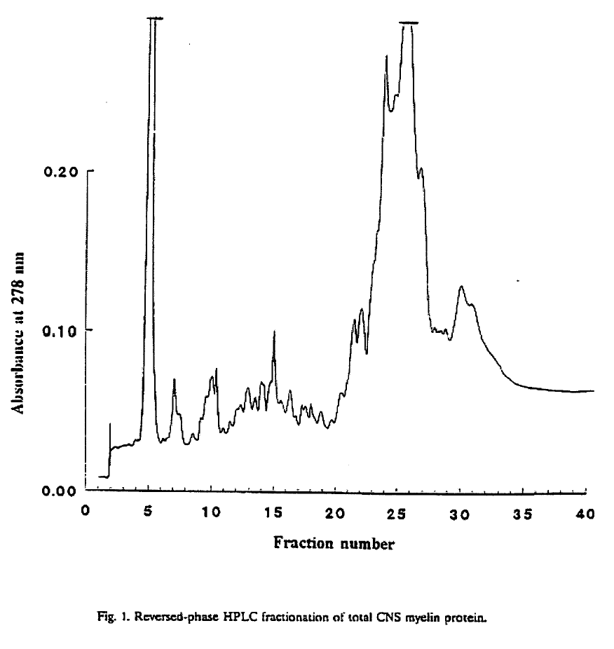

Although the notion that a comprehensive comparison of all respective immunogenicities of myelin proteins has long been warranted, its technical implementation has always posed serious problems. A major problem has been the fact that most of the myelin proteins, being membrane proteins, are very hydrophobic and poorly soluble in aqueous solution. Yet, solubility in aqueous environment is a strict prerequisite for performing immunological assays on these proteins. We have recently circumvented the problems associated with poor solubility by combining exhaustive protein delipidation with a quantitative two-step transfer of all CNS myelin proteins from organic solvents to aqueous solutions without losing their solubility [13]. As a result, we have been able to make all proteins available for immunological studies. Also, delipidated myelin proteins could be separated by reversed phase HPLC with a resolution beyond what had been reported sofar (see FIG. 1). In this way, a comparative study could be performed in which helper T cell responses were assayed in response to separated myelin proteins after priming of the T cells with all CNS myelin proteins in a single preparation.

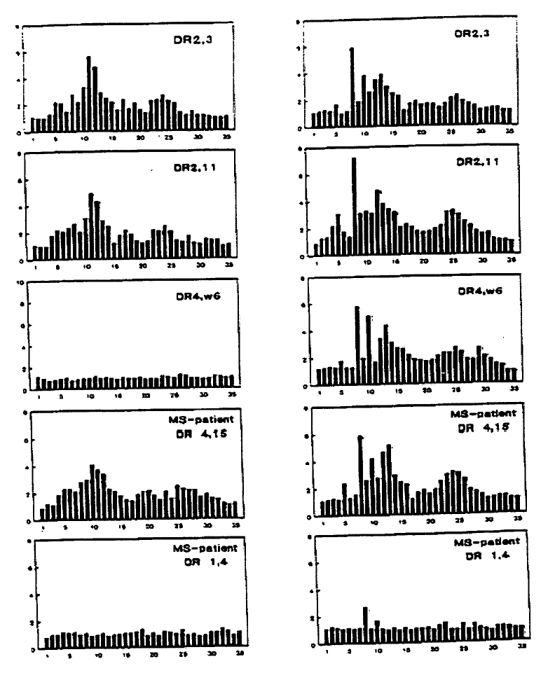

Moreover, the examination was performed in parallel experiments using human myelin proteins from MS-affected brains in one set of tests and myelin preparations from control brains in another. Using these sets of priming/test antigens, peripheral blood T cells from twenty-seven different individuals have been tested for proliferative responses to the fractionated myelin proteins. This novel approach has yielded unexpected and very useful data. They are represented in FIG. 2 (given on a separate sheet).

First, helper T cells from all donors were found responsive mainly to minor myelin proteins whereas only very weak responses were detected to either of the two predominant components MBP or PLP. This low response to the major protein components was found with T cells derived from both healthy and MS-affected donors. Secondly and most importantly, a surprising difference was observed in responses against myelin proteins isolated from MS-affected brain. Only when using this set of proteins as test antigens, a predominant proliferative T cell response turned out to be directed at a single HPLC protein fraction in all cases examined. T cell samples from healthy donors and MS-affected ones consistently showed the highest response to this particular protein fraction.

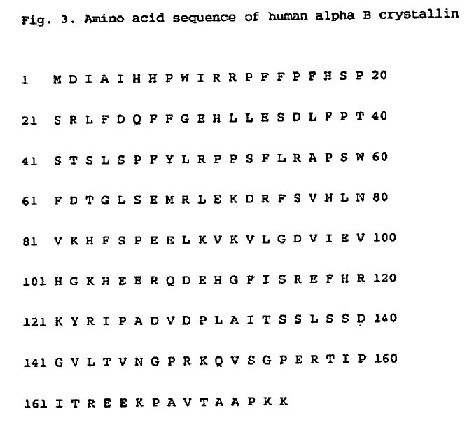

Close examination of this protein fraction revealed that it almost exclusively contained a 23 kDa protein which, upon sequencing, was identified as human alpha B crystallin. Although direct sequencing of the purified protein was impossible due to an N-terminal modification, six tryptic peptides derived from it consistently yielded sequences identical to alpha B crystallin (Table 1.). The protein contains 175 amino acids; only four amino acids are different between the human and the bovine form of the protein. The amino acid sequence of human alpha B crystallin is given in FIG. 3.

Alpha B Crystallin as Autoantigen in MS

Until 1989, crystallins including alpha B crystallins were believed to be proteins exclusively located in the eye lens.

Upon screening different tissues with antisera against alpha B crystallin, however, its presence was also established in striated (skeletal) muscle, kidney and CNS tissue [14].

Within the normal CNS, alpha B crystallin is mainly found in oligodendrocytes located in the white matter. Interestingly, previous studies on alpha B crystallin have revealed that its sequence is highly homologous to that of the family of small heat-shock proteins (or stress proteins) [15]. Consistent with its role as heat shock protein, alpha B crystallin has been found to exhibit chaperone characteristics [16].

In addition, immunohistochemical studies have confirmed that cellular levels of alpha B crystallin increase as a result of stress. In a number of pathological conditions including e.g. Alzheimer's disease and Alexander's disease levels of alpha B crystallin in the CNS have been found to be elevated [17, 18]. In the latter condition, significant amounts of alpha B crystallin are found to be accumulated inside astrocytes in the form of fibres, so-called Rosenthal fibres. Our own preliminary data obtained by immunohistochemical staining of CNS tissue with polyclonal anti-alpha B crystallin antibodies indicate enhanced expression within or closeby MS lesions as compared to unaffected regions of white matter. Thus, not only sequence homologies but also actual tissue expression of alpha B crystallin point to a role of this protein as heat-shock protein.

The combination of immunodominant target antigen among all different myelin proteins and its role as heat shock protein within glial cells renders alpha B crystallin a highly attractive candidate autoantigen. Our observation that only when using MS-affected myelin preparations a clear T cell response is found to the protein may well be explained by increased levels of the protein in such preparations relative to control preparations. However, particular diseaseassociated modifications of the protein may still be considered relevant as well. It is known that alpha B crystallin may be the target of various modifications including e.g. phosphorylations. If alpha B crystallin is the autoantigen in MS, its role as heat shock protein may explain why epidemiological studies on MS have pointed to a role of viral infections in the development of MS despite the fact that no single type of virus has been identified which has consistently been found to be associated with MS [19].

Viral infections within the CNS, albeit involving different types of viruses, may readily lead to a common side effect viz. enhanced local expression of alpha B crystallin as the result of stressful inflammatory mediators. At the same time, local infection will result in a local increase of class

II MHC molecules e.g. on infiltrating macrophages or on resident astrocytes [20]. It is not difficult to envisage that the combination of enhanced expression of antigen presenting class II MHC molecules and increased levels of immunodominant alpha B crystallin at the same time and in the same location may result in passing a threshold for triggering alpha B crystallin-directed helper T cell responses. Also the perpetuaLion of such responses is easily explained along the same lines. The immunodominance of alpha B crystallin has been observed in all human HLA-DR backgrounds examined sofar, including the DR2 type that is associated with MS in Western Europe.

Diagnosis and therapy in MS based on alpha B crystallin as autoantigen.

The new and surprising finding that indeed, a single immunodominant myelin protein exists for all human class II MHC backgrounds and that this protein has both structural and functional characteristics of a heat shock protein as well, opens a series of possibilities for new approaches in diagnostics and therapy in auto-immunity related diseases, especially MS.

The main relevance of the identification of a dominant autoantigen in human autoimmune diseases is that such identification provides the key for implementing therapeutic approaches aimed at selective and long-lasting immune tolerization. The activation of certain sets of autoreactive memory T cells is crucial in triggering the damaging inflammatory process in human autoimmune disease including MS. Therefore, inactivation or enhanced control of these T cells is expected to be beneficial. Inactivation of harmful, autoreactive T cells can only be implemented in a selective manner when the antigen-specificity of the T cells involved is known. Without this knowledge, T cells can only be modulated in a general immune suppressive fashion, with inevitable negative side effects.

Several methods already exist for antigen-specific tolerance induction (see below). Yet, no single protein antigen has been identified in literature so far, that has been found useful as a tolerogen in human autoimmune disease including MS. The fact that no unique autoantigen has been identified in human autoimmune disease has led some researchers to hypothesize that in human chronic autoimmune disease, a multitude of different autoantigens fuel the process, rather than a single one. This idea is referred to as the concept of determinant spreading (reviewed by Miller et al, ref 1); once disease has started, many protein antigens would become target of the response. It is very important, however, to point out that no data on human autoimmune responses have been published so far to support this idea (not in 1994, and still not in 2000). In contrast, a large body of evidence indicates that antigen-specific autoimmune repertoires of patients that are suffering from autoimmune disease are—without any exception known to date—similar to autoimmune repertoires of healthy control subjects. For MS, data on this issue have been reviewed by Martin and McFarland in 1995 [2]; no data have been reported since that alter their conclusion. If determinant spreading would occur in human autoimmune disease, autoimmune repertoires could not be similar between patients and controls, but responses in patients would be detectably different from healthy controls. Also anti-alpha B-crystallin responses are similar in MS patients to those in healthy controls (present application). The autoimmune repertoire as such is therefore the same between autoimmune patients and healthy subjects, but once certain tissues become sites of antigen presentation, certain key antigens in the tissue may still predominate as targets of the response. In patients, the antigens are locally presented to T cells in conjunction with co-stimulatory signals (see below) while in healthy control subjects, they are not. The difference between patients and healthy controls would thus not be a different immune repertoire, but an intrinsically diseased target tissue that starts to present antigens a healthy tissue would not.

Based on the data in this application that clearly show a dominant T-cell response to alpha B-crystallin in CNS myelin from MS patients relative to the T-cell response to all other myelin-associated protein antigens, antigen-specific tolerance induction can be implemented to the expected benefit of MS patients. Methods to do so have been the subject of many studies (summarized below) and these studies have shown antigen-specific tolerance induction to be successful in experimental animal models. In fact, several of the methods have been the subject of previously granted patents that claim the use of specific proteins for tolerization in autoimmune disease [3-8; see also references and background information in these patents]. Importantly, these patents do not mention nor cover the application of alpha B-crystallin as a tolerogen in autoimmune disease. Two basic approaches have been described.

The most classical way to induce antigen-specific tolerance in mammals is oral tolerance induction [9]. It has been known for decades that ingestion of proteins promotes immunological tolerance that is selective for these ingested proteins. The mechanism by which tolerance is induced, however, is less apparent. It has been hypothesized that antigen introduced in the gastro-intestinal tract triggers activation of a specific set of regulatory T cells that actively down-regulate immune responses to that antigen. T cells secreting transforming-growth-factor-β have been proposed as main effectors of such a regulatory activity [10].

Alternatively, oral antigen may trigger the death of T cells that were designed to recognize them [11]. In order to understand how this may work, it is important to appreciate that activation of resting T cells requires multiple signals from the environment [12, 13]. Encounter with the right antigen is one of these signals, but other signals are equally essential to trigger resting T cells to come into action, regardless of the nature of the antigen. These additional, so-called co-stimulatory signals can be given by the appearance of stress-inducible surface molecules on activated antigen-presenting cells, or by soluble factors such as hormones, cytokines, or bacterial or viral products that ligate with receptors on the surface of T cells. Only when a T cell encounters the combination of co-stimulatory signals and the right antigen, it will proceed to stages of activation including proliferation and cytokine production. Interestingly, when a resting T cell encounters only antigen, but none of the other signals, it may be partially triggered to anergy (functional unresponsiveness) or apoptosis (programmed cell death) rather than productive activation [12-16]. Therefore, in the absence of co-stimulatory signals (such as is the routine state of a healthy gastro-intestinal tract) antigens alone will trigger the death of specific T cells rather than their activation.

There are no indications that this basic requirement for co-stimulation by resting T cells, or the mechanism for antigens to trigger either T-cell activation or T-cell death, are selective for certain antigen-specific T cells. Instead, they are based on general features of T cells and apply to all T-cell antigens alike. Therefore, the mechanisms for tolerance induction that rely on these general features of T cells are expected to be the same for all antigens, and they are expected to be the same in humans, rodents, primates and other mammals. In line with this view, effective tolerance induction has been described for a wide variety of orally administered antigens in different animal species, including for example myelin basic protein, proteolipid protein, acetyl choline receptor, influenza virus protein, ovalbumin, cytochrome c, retinal antoantigens, eye lens beta-crystallin and streptococcal cell wall proteins [17-26]

A variety of other ways have been developed to introduce antigens experimentally into mammals in such a way that the conjunction of antigen and co-stimulatory signals is prevented. Key to these approaches is the notion that co-stimulatory signals are usually indicative and reliant on cellular stress or damage or the presence of abnormal of toxic substances [27]. Thus, in normal healthy tissues, co-stimulatory signals are usually very low or absent. When antigen is introduced in a healthy and unstressed environment, therefore, tolerance induction is much more likely to result than T cell activation. An early example of tolerance induction based on this notion was the introduction of antigen in incomplete Freund's adjuvant in rodents [28]. While complete Freund's adjuvant contains bacterial products, potent inducers of co-stimulatory signals, incomplete adjuvant does not contain these products and accordingly, it is much less effective in triggering T cell reactions. Instead, potentially responsive T cells only meet with their antigen and are triggered to enter a stage of anergy or apoptosis when the antigen is given in incomplete Freund's adjuvant. Intravenous administration or intranasal administration of pure and clean solutions of protein antigen similarly leads to T cells encountering only antigen in the absence of co-stimulatory signals. Again, the predominant result is tolerance induction. Many examples have been described that illustrate the successful use of these therapeutic approaches in installing antigen-specific tolerance in rodents, and preventing experimental autoimmune disease in these animals.

Finally, a issue that deserves special discussion is the question whether or not antigen-specific tolerance induction can be therapeutically applied in ongoing disease. In human autoimmune disease such as MS, we are likely to be dealing with a situation in which memory T cells rather than naïve T cells are the main pathogenic population. As a mimic to this situation, tolerization experiments have been performed and published by several groups that involved tolerance induction applied only after the onset of experimental autoimmune disease[18, 21, 25, 29-31]. Indeed, also in such a situation, several tolerance-induction protocols have been found effective in ameliorating ongoing disease in animal models. Our own experimental results (see experimental section) confirm the idea that also with alpha B-crystallin as an experimental antigen, oral tolerance induction can be successfully established even after an antigen-specific priming event. Thus, methods for tolerance induction as described above are likely to be not only effective in preventing the start of autoimmune disease, but also in preventing the perpetuation or further development of in ongoing chronic disease

An Experimental Animal Disease Model for MS Based on Alpha B-Crystallin Autoreactivity?

The most appealing way to test therapeutic approaches to tolerance induction would be in an experimental disease model in laboratory animals. In such models, not only T-cell reactivity can be monitored, but also clinical disease. The impact of therapeutic intervention can then be assessed in vivo, at both levels. The model of experimental allergic encephalomyelitis (EAE) is a classical experimental disease model for MS, with clinical and histopathological features similar to MS. Routinely, it is induced by immunizing rodents or primates with myelin antigens in combination in strong adjuvants. Since EAE has been described as the result of immunization with a large variety of different myelin antigens (proteins as well as peptides) one would expect myelin-associated alpha B-crystallin—being such a dominant T-cell activator- to also be encephalitogenic in rodents. Studies performed in our group since the first patent application in 1994, however, have now clarified that an unusual situation exists with regard to alpha B-crystallin. In humans, the self protein triggers strong T-cell reactivity, consistent with studies that have shown the protein to be absent from all healthy lymphoid organs and tissues [32]. Not being used to the protein, therefore, (even despite it is a self protein), the human immune system strongly responds to it when challenged. In other mammals including rodents and primates, however, alpha B-crystallin is a constitutive protein component of secondary lymphoid organs such as spleen en lymph nodes, and frequently also of primary lymphoid organs such as the thymus [32-35]. Consequently, the immune system in these animals is naturally tolerant for the self protein: they are continuously confronted with the protein in healthy lymphoid cells and tissues. Functional T- and B-cell nonresponsiveness to self alpha B-crystallin (but not to foreign alpha B-crystallin) has been unequivocally demonstrated for Lewis rats [33] and SJL mice [35], two rodents strains that are quite susceptible to EAE induction with other myelin antigens. Only in a mouse strain that does not express alpha B-crystallin in the thymus (Biozzi AHB mice) [32], some T-cell responsiveness could be induced by harsh immunization protocols, and low levels of clinical and histological EAE could be induced as well with a peptide fragment of alpha B-crystallin [34]. Together, these data indicate that while alpha B-crystallin specific T cells can in principle trigger CNS inflammation and clinical disease, the rule in mammals other than humans is functional non-responsiveness to self-alpha B-crystallin at the level of T and B cells. Only in humans, the protein is absent from the lymphoid compartment, and becomes a target of autoimmune responses when it is functionally presented in the CNS, as we know it is in early MS lesions [36, 37].

Studies of tolerance induction using alpha B-crystallin can therefore not be performed in normal rodents since these animals are already tolerant in their normal state. In order to study tolerance, we therefore moved to the use of alpha B-crystallin-deficient mice. By virtue of the absence of alpha B-crystallin from lymphoid cells, these animals mount vigorous responses to self (murine) alpha B-crystallin when this antigen is injected. Natural responsiveness to self-alpha B-crystallin therefore seems to be a feature that is unique to humans, an interesting finding in the light of the fact that also MS is only known to occur in humans and does not have a natural spontaneous counterpart in other animals.

It is important to note that while the actual situation leads to humans being responsive and other animals being tolerant for self-alpha B-crystallin, the general rules of tolerance induction as described above appear to fully apply to alpha B-crystallin as they do to other antigens. Immune responsiveness and non-responsivness to self-alpha B-crystallin in different species perfectly matches lymphoid absence and presence of the protein, respectively. This is fully in line with the basic rules of tolerance that apply also tor other antigens. The apparent differences between humans and other mammals, therefore, does not imply different rules for tolerance induction for alpha B-crystallin between these species.

Diagnostics in MS has always been a problem. Not only the intrinsic variety of different clinical symptoms that may be caused by local afflictions in the CNS, also the time course of clinical signs considered to be characteristic to MS renders a rapid diagnosis virtually impossible. If relapsing remitting phenomena are considered to be of crucial importance in establishing the diagnosis, one will simply have to monitor disease for an extended period of time. The recent advent of magnetic resonance imaging as a diagnostic tool in monitoring local CNS inflammations has contributed greatly to diagnostics in MS but has not yet completely solved the time problem. A lengthy procedure for the diagnostic confirmation of MS is undesirable, not only for the MS patients themselves but also for the possible efficacy of therapeutic approaches. Specific immune responses to alpha B crystallin may assist in diagnosing MS.

First, the formation of immunoglobulins, specific to alpha B crystallin, may be assessed in tissues and/or body fluids such as blood or cerebrospinal fluid and they may be specifically enhanced in MS. Such enhanced antibody responses may be employed as a diagnostic marker for MS in the design of antigen-based kits to monitor antibody responses.

Antigen based kits should be read to include, but not be limited to ELISA's, RIA's, SPIA's and for DIA's (differing foremost in the kind of label attached to the specific binding reagent for the antibody to alpha B crystallin) in any suitable format such as sandwich-, competition- or agglutination-assays.

A suitable format is a sandwich-assay, wherein alpha B crystallin is provided on a solid phase, whereby a sample suspected of containing the antibodies to alpha B crystallin is contacted with said solid phase, so that it can bind to the antigen, after which the solid phase is contacted with labelled antigen, or with a labelled antibody directed to the alpha B crystallin antibody. Instead of whole antigens and/or antibodies it is of course very well possible to use specifically binding parts thereof.

Many labels are known and can be applied in assays according to the invention. Such labels include enzymes, which usually need a substrate to give a signal, particle sols, such as metal (gold) sols, coloured latex particles, dyestuffs, fluorescent materials and radioactive materials. The assay itself can be in a laboratory format, or for use in the Doctor's Office or even for home use. The man skilled in the art will be able to construct a suitable assay based on the invention.

Alternatively, T cell responses and in particular helper

T cell responses to alpha B crystallin may serve as diagnostic marker in MS. A variety of methods may be envisaged for the detection of augmented T cell responses such as screening for activation markers on alpha B crystallin-specific T cells in body fluids including cerebrospinal fluid, examining frequencies of specific T cells or enhanced production of cytokines in response to alpha B crystallin.

Thus, increased immune responses against alpha B crystallin, either humoral or cellular, which may be specifically associated with MS may be employed to assist diagnosis.

For the development of antigen-specific therapeutic approaches in MS, a large variety of strategies are already available, and others are yet to be developed [reviewed in 21]. Antigen-specific strategies have been tested in animal models of autoimmunity and several of these methods have been found effective in preventing or ameliorating disease. Such specific therapies may be aimed at intervention in the productive formation of trimolecular interactions between antigen, T-cell receptor and HLA molecule which result in activation of the T cell involved. Alternatively, antigen (or fragments of it) may be administered in such a way that specific tolerance rather than immune responsiveness is triggered. Thus, specific intervention may be achieved at different levels. The administration of antigen via toleranceinducing routes may be employed to mount tolerance to a specific autoimmunity-inducing antigen. For example, oral administration of target antigen may lead to a state of tolerance in which autoimmunity can no longer be induced. The same may be achieved by frequent administration of either very low or very high doses of antigen over an extended period of time. Either dosage may fail to induce immunity but, instead, may finally result in a state of non-responsiveness or tolerance to the antigen used. Also thymic implantation of antigen may result in specific tolerance since the thymic environment by itself appears to be designed to induce tolerance in T cells that locally respond to their target structure. These routes of administration designed to induce tolerance, or future alternatives for it, may be taken with whole antigen or selected fragments derived from it either as free entities or as part of a more complex structure (e.g.

encapsulated or expressed by micro-organisms).

Also, antigen can be administered together with agents that interfere with the co-stimulatory signals that are essential to T cell activation.

It has recently become clear that the recognition of antigen/HLA complexes by the T-cell receptor alone is not sufficient for productive activation of T cells. For this, additional interactions between several accessory molecules on the surface of antigen-presenting cells and complementary molecules on the surface of T cells are required. Recognition of antigen/HLA without these accessory interactions may not only fail to activate the T cell, it may even lead to a state of antigen-specific anergy (unresponsiveness) of the T cell [22]. This phenomenon may be employed to induce specific tolerance in man or animals [23]. For example, administration of antigen/HLA complexes (as free or membrane-bound preparations) may specifically anergize the antigen-specific T cells rather than activate them. Also the administration of antigen on B cells, which lack essential accessory molecules to activate naive (previously unactivated) T cells may have the same effect. Finally, specific antibody preparations (or other therapeutic agents) targeted at accessory molecules, when given in conjunction with antigen could also result in the anergy of T cells specific to the co-administered antigen.

It is to be expected that yet other strategies to the induction of antigen-specific anergy will be developed in the future but these will remain crucially dependent on knowledge of the antigen.

Unresponsiveness (or non disease-inducing responsiveness) may not only be achieved by a specific route of antigen administration, but also by administration of selected fragments of the antigen. In animal models of autoimmunity, it has been demonstrated that autoimmunity-inducing antigens may contain sequences which may prevent or ameliorate the development of autoimmune disease when given as separate entities from the intact antigen. Thus, treatment with selected fragments of antigen may have the opposite effect as treatment with whole antigen. An interesting variant to this concept is the administration of slightly altered sequences of the antigen. Antigen fragments that may normally be involved in the activation of autoreactive T cells may be altered somewhat in sequence in order to achieve the opposite effect: specific unresponsiveness of antigen-specific T cells. Such slightly altered antigen fragments (APL's or ‘altered peptide ligands’) may act as antagonists or modulator peptides. These may specifically engage TCR with an authentic specificity to the autoantigen in such a way that productive accessory interactions, required for T cell activation, are prevented.

The effect is similar to that in the case of preventing accessory signals by other methods: specific unresponsiveness of the T cell involved. Although it cannot be predicted beforehand which alterations in the antigen sequence may or may not produce such an ‘antagonistic/modulator’ effect, it seems likely that these effects are the strongest with sequences very similar to the authentic antigen sequence.

Another target for antigen-specific intervention in T cell mediated autoimmunity is the T cell itself. Just like the structure of antigen is unique to its identity, the structure of the T-cell receptor (TCR) is unique to the recognition of its antigen target. Thus, TCR structures may be identified that are specific to the recognition of a given antigen. These structures can be found in the variable-, joining- or diversity-coding regions of the TCR gene for either alpha or beta chain. Structures specific to the recognition of a given antigen may be designated clonotypic or idiotypic TCR structures. Again, experiences in animal modes for autoimmunity have demonstrated possibilities for intervention in disease by specifically aiming intervention at these clonotypic/idiotypic TCR structures. First, this may be achieved by vaccination with antigenspecific T cells themselves. T cells to any antigen may be selected by culturing in vitro and vaccination may be carried out with inactivated populations of such presumed autoreactive T cells.

Vaccination is aimed at raising an immune response (either cellular or humoral) in the diseased individual specific to the antigen-specific T cells themselves in order to achieve their elimination from the body. Secondly, the same may be achieved by vaccination strategies employing not the complete T cell as therapeutic agent, but only relevant structures such as the TCR alone or even just small sequences corresponding to clonotypic or idiotypic determinants in the

TCR. For such strategies, TCR or sequences derived from it may also be used as part of larger structures. Although not yet demonstrated, antigen-specific (idiotypic) features of T cells may. not only be reflected in structural elements of the TCR alone but could in principle also be reflected in alternative specific structures either within or on the surface of cells.

Development of antigen-specific therapies in autoimmunity has been an elusive goal for many years. Advances in experimental animal models where trigger autoantigens are known by preselection have shown an increasing number of successful approaches to prevent even ongoing autoimmunity [21]. Either the antigen structure itself or features of the specific T cells activated by it may be employed for the rational design of therapy. Also in human autoimmunity, identification of all idiotypic and other relevant features of autoreactive T cells that could be involved in auto immunity is fully dependent on identification of the relevant antigen. In other words: the identification of the antigen is the key to selection and characterization of the responding T cells.

Also the assessment of genetic predisposition to MS may be assisted by an analysis of alpha B crystallin gene sequences in the human genome. The possible occurrence of polymorphisms within the alpha B crystallin gene or adjacent sequences on human chromosome 11 may provide clues to altered (enhanced) expression or modified protein structures, both of which could be relevant to the capacity of the gene product to trigger autoimmune responses and thus, to the likelihood of developing MS.

Alpha B crystallin does not only provide a structural trigger/target for autoimmunity in MS, limited to white matter in the CNS, it may also provide an explanation of how tissuespecific autoimmunity against CNS myelin may have a multifactorial onset [19]. Behaving as heat-shock or stress protein, alpha B crystallin may locally be expressed in high levels in response to a variety of challenges such as persistent neurotropic infections, ischaemic attacks or physical injury. Together with its immunodominance to human T cells, increased levels of alpha B crystallin may occasionally be expected to raise beyond the threshold for T cell activation, especially if e.g. infections at the same site raise local class II MHC expression of microglia and astrocytes. This notion may provide the basis for yet another approach to therapy, i.e. aimed at reducing alpha B crystallin levels in the CNS. Molecular biological approaches involving for example antisense DNA or RNA may be developed in order to specifically block or reduce expression of alpha B crystallin in the target tissue. Such goals may be achieved e.g. by gene therapy or by infection with altered viruses that produce antisense nucleic acids or so-called “hammerhead” ribozymes directed at destroying the alpha B crystallin messenger or reducing its levels. It may be envisaged that in the future also other strategies may be developed along this line that are based upon the identification of alpha B crystallin as target autoantigen.

Thus the invention provides an important major autoantigen (alpha B crystallin) for use in the diagnosis or therapy of auto-immunity related diseases, in particular Multiple Sclerosis. It will be clear from the afore going that alpha B crystallin can be used in many ways according to the invention. It is, however, also true that it may not be necessary to use complete alpha B crystallin.

In many instances it will also be possible to apply only part of the whole protein or derivatives of the protein and the like, which have similar activity as alpha B crystallin.

Accordingly these parts and/or derivatives are also part of the present invention.

For diagnosis it has been explained hereinabove how assays can be carried out and how test kits can be assembled on the basis of the alpha B crystallin.

For therapeutic purposes, for instance when the antigen is to be administered in a composition for induction of tolerance it will be clear that depending on the route of administration (parenteral, enteral) and the patient the dosages may vary. Generally they will lie between 1 ng/kg body weight/day and 1 mg/kg body weight per day or other efficacious doses.

Such compositions may of course comprise suitable excipients.

How the alpha B crystallin is obtained is not important. It may be isolated from myelin material or other tissues or it may be produced recombinantly through suitable host cells.

Fragments (peptides) may be produced synthetically.

For recombinant production it is necessary to provide the gene coding for alpha B crystallin, which can be easily done because the amino acid sequence is known.

For instance techniques like PCR will be very helpful in this respect. Having the gene provides yet other useful diagnostic and therapeutic tools according to the invention.

It will be clear that PCR and NASBA and the like can be used to diagnose enhanced expression of alpha B crystallin.

The usefulness of antisense approaches has already been mentioned before. For these approaches it will also be clear that it is not always required to use the entire gene or an unaltered gene. Fragrnents and/or derivatives may also be used.

For expression of the gene many cells, vectors and expression regulators such as promoters, enhancers and the like are known. If post-translational modifications are necessary or if a signal sequence needs to be removed, it may be best to employ eukaryotic cells such as CHO cells and the like.

It will be clear for the man skilled in the art that it is also possible to obtain derivatives of alpha B crystallin, which will work as antagonists and thus block the T cell response to alpha B crystallin. Another way of arriving at such antagonists is by providing anti-idiotype antibodies, which have a complementarity determining region which is almost an internal image of the epitope of alpha B crystallin recognized by the T cell receptor. It is also possible to identify the responsible T cell receptor and prepare antibodies thereto.

These and any other antibodies may be prepared according to any of the well known techniques. Preferably monoclonal antibodies are prepared. It is usual practice to produce antibodies in rodents, however, if they have to be applied in humans, these will usually lead to immuneresponse.

Techniques to reduce the immunogenicity of these antibodies by providing fragments or hybrid human/rodent antibodies, as well as techniques to engineer these antibodies into humanized antibodies (CDR-grafting) are all within the art.

As stated herein before it is also possible to prepare vaccines based on the responsible auto-reactive T cells. For this purpose the T cells have to be inactivated.

Instead of complete T cells their receptors (TCR's) or parts of derivatives can be used. They can, like the antigens and the antibodies, be obtained in a variety of ways, including isolation, synthesis, genetic engineering, etc.

Any and all vaccines can comprise the usual excipients.

The invention will now be explained in greater detail in the following examples.

Experimental Section 1. Materials and Methods 1.1 Preparation of Myelin Proteins for Immune Assays

White matter samples from human brains obtained by rapid autopsies were kept frozen at −800 C until use. Samples including white matter lesions were obtained from 13 definite MS patients and another set of samples from 21 control subjects. CNS myelin was purified from white matter samples as described by Norton and Poduslo [24]. Purified myelin preparations from either MS patients or control subjects were pooled separately and stored lyophilized at −200 C until use.

For the preparation of whole myelin protein as stimulatory antigen, purified myelin was delipidated according to van Noort et al. [13]. Briefly, myelin was dissolved in 80% (v/v) tetrahydrofuran, 20% (v/v) water, 0.1% (v/v) trifluoroacetic acid and passed over Sephadex LH-60 in order to separate proteins from (glyco)lipids. Protein-containing eluate was pooled and the proteins were precipitated from the solution by adding diethylether. Precipiated proteins were spun down by centrifugation and lyophilized. Delipidated myelin proteins were subsequently dissolved in 2-chloroethanol containing 0.1% (v/v) trifluoroacetic acid at a concentration of approximately 4 mg/ml and dialyzed against four changes of water in regenerated cellulose acetate dialysis membranes. The final protein concentration in the fully aqueous solution was determined by amino acid analysis. The aqueous solutions of total myelin proteins were kept at +40 C since any freezing of such preparations leads to irreversible precipitation of the more hydrophobic protein molecules.

Fractionation of all myelin proteins for use as test antigen in the assays was performed as described previously by van Noort et al [13; see FIG. 1] using a reversed-phase column with a C3 matrix (Beckman Instruments, San Ramon, Calif., USA).

Following collection and lyophilization of 40 protein fractions as obtained by HPLC, the contents of each fraction were dissolved in 2-chloroethanol, 0.1% trifluoroactetic acid and dialyzed against water as described above. All aqueous protein fractions were kept at +40 C until use. Based upon amino acid composition analyses of protein samples, equal amounts of total protein mass were taken from both MS-affected and control myelin samples for HPLC fractionation and testing in the proliferation assays.

1.2. Testing of Bulk T-Cell Responses to Myelin Proteins.

Peripheral blood mononuclear cells (PBMC) were obtained by lymphophoresis from twenty-seven HLA-typed donors amongst which five patients with definite MS according to the Poser criteria. PBMC were isolated according to routine procedures and stored at −1960 C until use.

PBMC from each donor were grown at 370 C in a humidified stove under a 5% CO₂-atmosphere in 96-well round-bottom microtiter plates. PBMC were seeded at 2.105 cells per 200 Fl in RPMI1640 culture medium (Dutch modification) supplemented with 10 mM HEPES, 2 mM glutamine, 1 mM sodium pyruvate, 0.1 mg/ml streptomycin, 100 U/ml penicillin and 10% (v/v) pooled human serum in the presence of total myelin proteins at a final concentration of 25 Fg/ml. After seven days, 100 Fl of supernatant medium was removed and growing T cells were restimulated by adding 100 R1 of fresh RPMI 1640 culture medium containg 1.105 irradiated (30 Gy) autologous PBMC and fresh total myelin protein at a concentration of 50 Fg/ml. After eleven days, human recombinant IL-2 was added to a final concentration of 50 U/ml. After fourteen days, cells were collected and tested in triplicate for proliferation against HPLC-fractionated proteins in a standard proliferation assay.

A fixed sample of the contents of each HPLC fraction was added to wells containing 5.104 cultured T cells and 5.104 irradiated autologous PBMC in 200 Fl culture medium.

After three days, 0.6 RCi [3H]-thymidine was added to each well and following another 18 h, cells were harvested and thymidine incorporation was determined using a betaplate counter.

By using the above approach, the protein concentration in each well during the proliferation assay varied according to the protein contents of the corresponding HPLC fraction. For those fractions containing either MBP or PLP, the predominant proteins of myelin, final protein concentration in the well was calculated to be about 50 Fg/ml on the basis of amino acid analysis of a sample from the original HPLC fraction. Total protein concentrations of HPLC fractions that contain minor proteins were about five- to ten-fold less. The effects of protein concentration upon the qualitative aspects of the result were examined by testing proliferative responses to dilutions of HPLC fractionated proteins.

The results obtained were consistent with the dilutions used and revealed no anomalies (data not shown).

1.3 Purification and Identification of Alpha B Crystallin

The results obtained in the proliferation assays revealed consistent and strong proliferative responses to one particular protein fraction derived from MS-affected myelin (see below under “Results”). A sample of this fraction, designated by sequential numbering HPLC fraction 8, was analyzed by SDS-polyacrylamide gelelectrophoresis as well as by additional reversed-phase HPLC. Re-chromatography by HPLC was performed by using another type of reversed phase HPLC column, viz. a mixed C1/C8 matrix (Pharmacia LKB, Bromma, Sweden) instead of a C3 matrix (Beckman Instruments, San Ramon, Calif., USA) and another eluent, viz. acetonitrile instead of a tetrahydrofuran/acetonitrile mixture.

As described under “Results”, both SDS-PAGE and RP-HPLC analysis of the contents of the highly immunogenic fraction 8 revealed the almost exclusive presence of a protein with an apparent mass of approximately 23 kDa. Using a novel preparation of MS-affected myelin as a source, this 23 kDa protein was purified to apparent homogeneity by sequential RP

HPLC using a C3 matrix and tetrahydrofuran/acetonitriLe as an eluent in the first step and a mixed C1/C8 matrix and acetonitrile as an eluent in the second and third steps. Step three differed from step two in the gradient applied; 0.1% (v/v) trifluoroacteic acid was used as a solvent throughout.

The purified 23 kDa protein was examined by SDS-PAGE and analyzed by amino acid composition analysis. The protein was also subjected to amino acid sequencing using an Applied

Biosystems 470A Model on-line equipped with an Applied

Biosystems Model 120A PTH amino acid analyzer. In order to generate fragments of the 23 kDa protein for sequencing, 100 pig of purified 23 kDa protein was dissolved in 100 pil 50 mM Tris-HCl pH 7,8 and supplied with 1 pig bovine spleen trypsin. After 24 h at 370 C, the tryptic digest was fractionated by RP-HPLC on a C 18 matrix column using a gradient of acetonitrile in 0.1% (v/v) trifluoroacetic acid.

Individual fragments were purified by additional RP-HPLC using 50 mM ammoniumacetate pH 5.8 as a solvent and acetonitrile as an eluent.

For the generation of a polyclonal antiserum against the immunogenic 23 kDa protein, a rabbit was immunized twice with 20 pig of purified 23 kDa protein emulsified in complete Freund adjuvant (CFA) and a third time with 100 pig of protein in CFA at six-week intervals. By using western blotting according to standard procedures, the antibodies raised were tested on recognition of SDS-PAGE separated contents of the original

HPLC fraction 8, of the corresponding fraction derived from control myelin, on purified 23 kDa protein used for the immunization, on purified MBP and, finally, on purified alpha

B crystallin and alpha A crystallin from bovine eye lens. Both A and B chain of alpha crystallin from bovine lens were purified from a commercial preparation of alpha crystallin (Sigma Chemical Co, St. Louis, Mo., USA) by reversed phase

HPLC. Prior to application onto a reversed phase column, alpha crystallin was dissolved in 8 M urea, 10% acetic acid and kept at 37° C. for 1 h in order to completely dissociate A and B chains. Using a mixed C1/C8 reversed phase matrix, application of a gradient of acetonitrile in 0.1% (v/v) trifluoroacetic acid results in the baseline separation of A and B chains.

1.4 Example: Therapeutic Application of Oral Alpha B-Crystallin to Reduce an Ongoing Antigen-Specific T-Cell Response

In multiple sclerosis (MS) patients, a memory T-cell repertoire exists against human alpha B-crystallin which is readily demonstrable by assaying the response to purified preparations of recombinant human alpha B-crystallin. When taken from peripheral blood, T cells immediately proliferate and they secrete cytokines (notably interferon-γ) in response to the antigen [36]. Immunohistochemical evidence shows that myelin-derived alpha B-crystallin is functionally presented to infiltrated T cells at the earliest stages of MS lesions [37]. At that stage, the response of human T cells to myelin-derived alpha B-crystallin alone is similar to the response to whole myelin membranes. It is this response that is considered the driving force behind the development of inflammatory lesions in white matter of the central nervous system that are key to MS. Reduction or abolishment of the existing memory T-cell response to alpha B-crystallin in MS patients is therefore expected to reduce or halt the progression of disease.

In the following example it is disclosed how human alpha B-crystallin can be administered in such a way that tolerance is induced, and an ongoing T-cell response is abolished. Three weeks after mice were immunized with alpha B-crystallin in complete Freund's adjuvant and, therefore, well after a T-cell response has been established, oral administration of alpha B-crystallin strongly reduces the T-cell response in both lymph nodes and spleen.

The experimental animals used for the example are alpha B-crystallin-deficient mice. In normal rodents, a T-cell response to alpha B-crystallin cannot be studied. As normal rodents constitutively express alpha B-crystallin in primary and secondary lymphoid organs, they are already functionally tolerant for the protein. In alpha B-crystallin-deficient mice (αB-knock-out, or αB-KO mice), this build-up of tolerance has not occurred and αB-KO mice mount a strong cellular and humoral immune response to alpha B-crystallin when it is immunized in complete Freund's adjuvant. In this respect, they mimic humans who also do not express the protein in lymphoid cells and, accordingly, also respond strongly to alpha B-crystallin.

2. Results

2.1 Proliferative Response of Peripheral Blood T Cells to Myelin Proteins

The short period of only two weeks for expansion of peripheral blood T cells in response to antigen was chosen to minimize selective pressure upon T cells growing in vitro as a result of the experimental protocol or the type of culture medium used. We have found two weeks the minimimum time required to expand sufficient numbers of cells for a significant proliferative response to antigen of the bulk population. In this way, the specificity of the response measured is kept as representative as possible for the full repertoire of T cells responsive to myelin proteins.

The results reveal that by using bulk T cells from peripheral blood as responder cells, significant variations between individual donors may occur. Ten out of twenty-seven samples tested yielded no useful data. Either no growth at all was measured or background proliferation was excessive.

Seventeen out of twenty-seven samples from different HLA-typed donors, however, gave useful results in revealing significant proliferation to some HPLC fractions as well as to the complete set of myelin proteins that were used as stimulatory antigen. Three out of five PBMC samples from MS samples patients displayed significant proliferation against myelin proteins. A representative set of data, illustrating responses obtained with five samples, is given in FIG. 2. In the left panel, responses are shown to HPLC-fractionated proteins isolated from control myelin. In the right panel, responses against proteins derived from MS-affected myelin are shown.

The qualitative aspects of the response profiles obtained against control myelin proteins were remarkably similar among the different donors, irrespective of HLA typing or whether or not the donor suffered from definite MS. In all cases, responses were predominant to proteins that elute along the gradient at positions intermediate between the highly hydrophilic MBP (fractions 5 and 6) and the very hydrophobic PLP's (fractions 22-28). Responses to the major proteins MBP and PLP were modest, if at all significant. These findings are in line with our earlier observations [13]. It remains to be established whether or not the apparently predominant responses to the ‘intermediate’ fractions 10-15 point to the presence of a single immunogenic component or whether they reflect accumulated responses to a multitude of different minor proteins present in these fractions.

Proliferative responses to the set of proteins isolated from MS-affected myelin are similar to those against control material in revealing significant responses to the ‘intermediate’ fractions 10-15 that contain many different minor myelin proteins. Yet, the highest proliferative response in all samples examined was consistently directed at a single HPLC fraction, viz. fraction 8. Also when supplied at five- or ten-fold dilution did the contents of fraction 8 trigger the highest proliferative response (data not shown). The responses to fraction 8 were dependent on priming with myelin since unprimed PBMC or PBMC primed with an irrelevant antigen (Influenza virus) showed no response. Responses to fraction 8, however, did not depend on priming with MS-affected material since PBMC primed with control myelin showed comparable responses.

It should be noted that no clear differences have been observed thus far in the overall specificity of peripheral blood T cells from either healthy controls or MS patients.

However, the analysis of antigen specificity as performed here provides no data as to precursor frequencies, pre-activation state, fine-specificity or receptor features of T cells in peripheral blood nor data on T cells elsewhere in the body.

Thus, myelin-directed T cell responses in MS patients may still be different from control subjects in a number of aspects that have not been addressed in the present study.

Apparently, MS-affected myelin contains one or more highly immunogenic minor myelin antigen(s) for peripheral blood T cells from donors of different HLA type including healthy as well as MS-affected individuals that is/are contained in HPLC fraction 8. Increased levels of such (an) immunogenic protein(s) in this fraction relative to the material obtained from control myelin most likely explains the striking difference observed between the two series of assays illustrated in FIG. 1. Below, the identification of this immunogenic protein is described.

2.2 The Immunogenic Protein in MS-Affected Myelin is Alpha B Crystalline

SDS-PAGE analysis as well as additional reversed-phase

HPLC analysis of the contents of HPLC fraction 8 containing

MS-afected myelin protein revealed the almost exclusive presence of a protein with an apparent mass of 23 kDa. Using a novel preparation of MS-affected myelin proteins as a source, this 23 kDa protein together with other proteins co-eluting at approximately the same position in the HPLC gradient were subfractionated by additional RP-HPLC steps. Again proliferative responses of peripheral blood T cells primed with whole myelin proteins were directed at those HPLC fractions that contained the 23 kDa protein.

The 23 kDa protein was purified to homogeneity by reversed-phase HPLC and subjected to direct amino acid sequencing. No clear signals, however, were recorded upon sequencing suggesting the presence of an N-terminal modification of the protein obstructive to sequencing. In order to allow sequencing of internal protein segments, tryptic fragments were generated from the 23 kDa protein. Six peptides were purified to homogeneity and subjected to sequencing. This resulted in the identification of three separate sequences; two pairs of peptides turned out to share their N-terminal sequence. All three sequences were identical to internal sequences of human alpha B crystallin (Table 1).

Imunoblotting using polyclonal rabbit antibodies raised against the 23 kDa protein purified from human MS-affected brain confirmed the identification of the protein as alpha B crystallin. Western blotting of the SDS-PAGE separated contents of HPLC fraction 8 from MS-affected myelin led to distinct staining only of the predominant 23 kDa protein band in this fraction. This protein band can also be detected in the corresponding HPLC fraction containing proteins from control myelin. Also the alpha crystallin B chain as isolated from a commercial preparation of alpha crystallin from bovine eye lens was recognized upon western blotting by the anti-23 kDa antibodies whereas no recognition could be detected of the

A chain of alpha crystallin nor of purified MBP from human MSaffected myelin. Co-migration upon SDS-PAGE was observed between alpha B crystallin from bovine eye lens and the purified 23 kDa human myelin protein from MS-affected myelin.

Also upon reversed phase HPLC, the purified 23 kDa protein and alpha B crystallin behaved identically.

TABLE 1

Tryptic fragments of purified human 23 kDa
protein contain alpha B crystallin sequences.

| | | | | | | | | | | | | | |
|---|---|---|---|---|---|---|---|---|---|---|---|---|---|
| fragment 1 | I | P | A | D | V | D | P | (L) | ... | | | | |
| α^{B} crystallin | I₁₂₄ | P | A | D | V | D | P | L | A | | | | |
| fragment 2: | Y | L | R | (P) | | | | | | | | | |
| α^{B} crystallin | Y₄₈ | L | R | P | | | | | | | | | |
| fragment 3: | A | P | S | W | F | D | T | G | L | S | E | M | R |
| α^{B} crystallin | A₅₈ | P | S | W | F | D | T | G | L | S | E | M | R |

2.3. Therapeutic Application of Oral Alpha B-crystallin to Reduce an Ongoing Antigen-Specific T-Cell Response

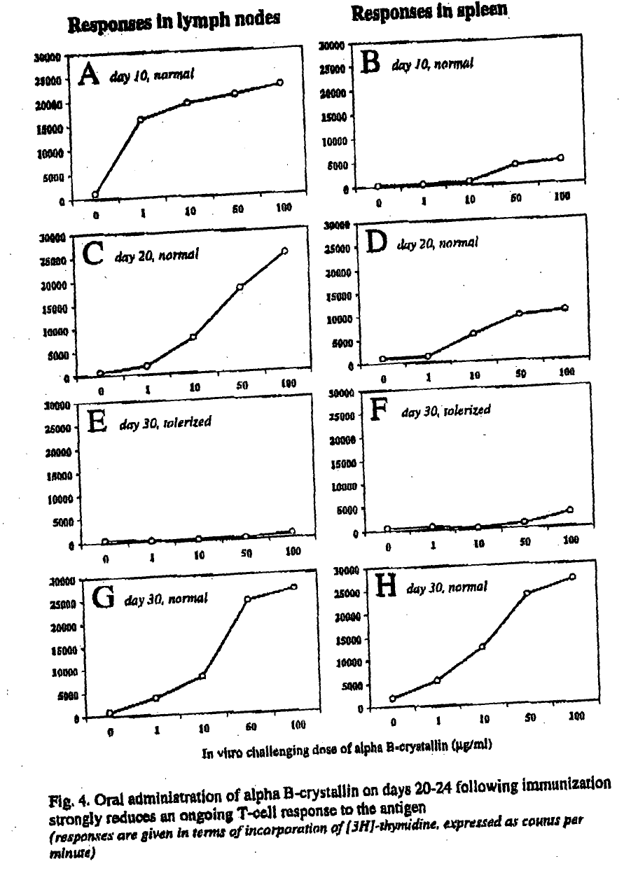

FIG. 4 shows the marked response by T cells derived from lymph nodes as well as from the spleen of αB-KO mice following immunization with alpha B-crystallin, as determined after 10 or 20 days (FIG. 4, panels A-D). When immunized animals are fed with recombinant alpha B-crystallin for five consecutive days, starting at day 20 following immunization, the antigen-specific response by T cells from lymph nodes and spleen, as assessed at day 30 is strongly reduced in the spleen and almost completely gone in lymph nodes (FIG. 4 panels E-F). In animals that have been fed with phosphate-buffered saline instead, no such reduction is observed and after 30 days, the antigen-specific response is still clearly detectable (FIG. 4, panels G-H).

These data provide compelling evidence that well after primary immunization of mice with recombinant human alpha B-crystallin, the antigen can be administered orally in such a way that the existing T-cell response is strongly reduced or abolished. Given the parallel response to alpha B-crystallin in both humans and αB-KO mice, this evidence indicates that also in humans, reduction or abolishment of an ongoing response to alpha B-crystallin should be achievable by oral administration of the antigen.

Experimental Details

Alpha B-crystallin-deficient mice were immunized with 2×50 μg recombinant human alpha B-crystallin emulsified in complete Freund's adjuvant. At day 10, 20 and 30 days following immunization, antigen-specific proliferative responses were determined for T cells derived from either lymph nodes or spleen using standard proliferation assays. Briefly, lymph node cells or spleen cells were collected and cultured with varying doses of antigen at 1×10⁵ cells per well together with 2×10⁵ irradiated splenocytes from naive mice as antigen-presenting cells in a total volume of 200 μl culture medium (RPMI 1640, Dutch modification, supplemented with 100 U/ml penicillin, 0.1 mg/ml streptomycin, 1 mM sodiumpyruvate, 50 mM 2-mercaptoethanol and 10% (vol/vol) fetal calf serum) at 37° C. in a humidified atmosphere containing 5% CO₂. After 72 h, 20 kBq [³H]-thymidine was added to the cultures. After another 18 h, [³H]-thymidine incorporation was measured using a beta-plate counter (Can berra Packard, Meriden Conn.).

Four groups of 5 mice each were used to monitor T-cell responses in lymph nodes (panels A, C, E and G in FIG. 4) and spleen (panels B, D, G and H) following a single immunization with alpha B-crystallin. The first two group were used to monitor the normal course of cellular responses at day 10 (panels A and B) and day 20 (panels C and D) following immunization. A third group were fed 2 mg recombinant human alpha B-crystallin dissolved in 250 μl phosphate-buffered saline on five consecutive days starting at day 20 following immunization. At day 30, T-cell responses were determined for this group (panels E and F). Animals in the fourth group were fed with a corresponding 250 μl phosphate-buffered saline without antigen, also for five consecutive days starting at day 20, before their T-cell responses were similarly determined at day 30 (panels G and H).

### REFERENCES

[1] Bjorkman P J M, Saper B, Samraoui B, Bennett W S, Strominger J L and Wiley D C (1987) Structure of the human class I histocompatibility antigen. Nature 329, 506.
[2] Weiss A (1991) Molecular and genetic insights into T-cell antigen receptor structure and function. Ann. Rev. Genet. 25, 487.
[3] Janeway C A (1992) The immune system evolved to discriminate infectious nonself from noninfectious self. Immunol. Today 13, 11.
[4] Sinha A A, Lopez M T and McDevitt H O (1990) Autoimmune disease: the failure of selftolerance. Science 248, 1380.
[5] Hafler DA and Weiner HL (1989) MS: a CNS and systemic autoimmune disease. Immunol. Today 10, 104.
[6] Raine CS (1991) Multiple sclerosis: a pivotal role for the T cell in lesion development. Neuropathol. Appl. Neurobiol. 17, 265.
[7] Lees M and Brostoff S (1984) Proteins in myelin. In: Morell P (ed) Myelin, Plenum, New York, pp 197-225.
[8] Van Noort J M, Van Sechel A C, Boon, J, Boersma W J A, Polman C H and Lucas C J (1993) Minor myelin proteins can be major targets for peripheral blood T cells from both multiple sclerosis patients and healthy subjects. J. Neuroimmunol. 46, 67.
[9] Campagnoni AT (1988) Molecular biology of myelin proteins from the central nervous system. J. Neurochem. 51, 1.
[10] Weiner HL and Hafler DA (1988) Immunotherapy of multiple sclerosis. Ann. Neurol. 3, 211.
[11] Wraith D C, Smilek D E, Mitchell D J H, Steinman L and McDevitt H O (1989) Antigen recognition in autoimmune encephalomyelitis and the potential for peptide-mediated immunotherapy. Cell 59, 247.
[12] Vandenbark AA, Hashim G, and Offer H (1989) Immunization with a synthetic T-cell receptor V-region peptide protects against experimental allergic encephalomyelitis. Nature 341, 541.
[13] Van Noort J M, El Ouagmiri M, Boon, J and Van Sechel A C (1994) Fractionation of central nervous system myelin proteins by reversed-phase high-performance liquid chromatography. J. Chromat. 653, 155.
[14] Iwaki T, Kume-Iwaki A, Goldman JE (1990) Cellular distribution of aB-crystallin in non-lenticular tissue. J. Histochem. Cytochem. 38, 31.
[15] Ingolia TD and Craig EA (1982) Four small Drosophila heat shock proteins are related to each other and to mammalian-crystallin. Proc. Natl. Acad.

Sci. USA 79, 2360.
[16] Nicholl I D and Quinlan R A (1994) Chaperone activity of a-crystallins modulates intermediate filament assembly. EMBO J. 13, 945.
[17] Iwaki T, Iwaki A, Tateishi J, Sakaki Y and Goldman JE (1993) aB crystallin and 27-kd heat shock protein are regulated by stress conditions in the central nervous system and accumulate in Rosenthal fibers. Am. J. Pathol. 143, 487.
[18] Iwaki T, Wisniewski T, Iwaki A, Corbin E, Tomokane N, Tateishi J and Goldman JE (1992) Accumulation of aB-crystallin in central nervous system glia and neurons in pathological conditions. Am. J. Pathol. 140, 345.
[19] Bernard CCA and Kerlero de Rosbo N (1992) Multiple sclerosis: an autoimmune disease of multifactorial etiology. Cuur. Opin. Immunol. 4, 760.
[20.] Fierz W, Endler B, Reske K., Wekerle H. and Fontana A. (1985) Astrocytes as antigen presenting cells. I.
   Induction of Ia antigen expression on astrocytes by Tcells via immune interferon and its effect on antigen presentation. J. Immunol. 134, 3785.
[21] Lanzavecchia A (1993) Strategies to specific immune intervention. Science 262, 45.
[22] Schwartz RH (1990) A cell culture model for T lymphocyte clonal anergy. Science 248,1349.
[23] Linsley P S, Wallace P M, Johnson J, Gibson M G, Greene J L, Ledbetter J A, Singh C and Tepper M A (1992) Immunosuppression in vivo with a soluble form of the CTLA-4 T cell activation molecule. Science 257, 792.
[24] Norton WT and Poduslo SE (1973) Myelination in rat brain: method of myelin isolation. J. Neurochem. 21, 749.
101. Miller S D, McRae B L, Vanderlugt C L, Nikcevich K M, Pope J G, Pope L, Karpus W J (1995). Evolution of the T-cell repertoire during the course of experimental immune-mediated demyelinating diseases. Immunol Rev 144:225-44
102. Martin R, McFarland H F (1995) Immunological aspects of experimental allergic encephalomyelitis and multiple sclerosis. Crit Rev Clin Lab Sci. 32(2): 121-82. Review.
103. Weiner H L et al., (1991) Enhancement of the down-regulation of autoimmune diseases by oral or enteral administration of autoantigens. EP 0594607. Int. Public. WO 91/12816
104. Weiner et al (1993) Treatment of multiple sclerosis by oral administration of bovine myelin. U.S. Pat. No. 5,849,298
105. Weiner et al. (1995) Treatment of multiple sclerosis by oral administration of autoantigens. U.S. Pat. No. 5,869,054
106. Holmgren et al. (1997) Immunological tolerance-inducing agent. U.S. Pat. No. 5,681,571.
107. Weiner et al. (1999) Treatment of immune diseases by oral administration of autoantigens. U.S. Pat. No. 5,869,093
108. McGuiness et al. (1999) Oral induction of tolerance to parenterally administered non-autologous polypeptides. U.S. Pat. No. 5,948,407
109. Weiner H L (1993) Treatment of autoimmune disease by oral tolerance to autoantigens. J. Autoimmun. 15, 6-7.
110. Hafler D A et al (1997) Oral administration of myelin induces antigen-specific TGFbeta 1 secreting T cells in patients with multiple sclerosis. Ann NY Acad Sci 19, 120-131.
111. Weiner H L et al (1994) Oral tolerance: immunologic mechanisms and treatment of animal and human organ-specific autoimmune disease by oral administration of antigen. Annu Rev Immunol 12, 809-837.
112. Schwartz R H (1996) Models of T-cell anergy: is there a common molecular mechnism? J Exp Med 184, 1-8.
113. Schwartz R H (1997) T cell clonal anergy. Curr Opin Immunol 9, 351-357.
114. Gimmi C D et al (1993) Human T-cell clonal anergy is induced by antigen presentation in the absence of B7 costimulation. Proc Natl Acada Sci USA 90, 6586-6590.
115. Guerder S, Flavell R A (1995) Costimulation in tolerance and autoimmunity. Int Rev Immunol 13, 135-146
116. Jenkins M K, Schwartz R H (1987) Antigen presentation by chemically modified splenocytes induces antigen-specific T-cell unresponsiveness in vitro and in vivo. J Exp Med 165, 302-319.
117. Higgins P J, Weiner H L (1988) Suppression of experimental autoimmune encephalomyelitis by oral administration of myelin basic protein and its fragments. J Immunol 140, 440-445
118. Barchan D et al (1999) Antigen-specific modulation of experimental myasthania gravis: nasal tolerization with recombinant fragments of the human acetyl choline receptor alpha subunit. Proc Natl, Acad Sci USA 96, 8086-8091.
119. Bercovici N et al (1999) Chronic intravenous injections of antigen induce and maintain tolerance in T-cell receptor transgenic mice. Eur J Immunol 29, 345-354.
120. Bitar D M, Whitacre C C (1988) Suppression of experimental autoimmune encephalomyelitis by the oral administration of myelin basic protein. Cell Immunol 112, 364, 370.
121. Brod S A et al (1991) Suppression of experimental autoimmune encephalomyelitis by oral administration of myelin antigens: IV. Suppression of chronic relapsing disease in the Lewis rat and strain 13 guinea pig. Ann Neurol 29, 615-622
122. Laliotou B et al (1999) Intranasal administration of retinal antigens induce transient T cell activation and apoptosis within drainage lymph nodes but not spleen. J Autoimmun 12, 145-155
123. Nicolle M W et al (1994) Specific tolerance to an acetylcholine receptor epitope induced in vitro in myasthenia gravis CD+lymphocytes by soluble MHC complex class II-peptide complexes. J Clin Invest 93, 1361-1369.
124. Sueno T et al (1997) Oral administration of lens homogenate suppress antibody production in mice injected with beta crystallin emulsified in CFA. Exp Eye Res 64, 379-385.
125. Thurau SR et al (1997) Oral tolerance in a murine model of relapsing experimental autoimmune uneoretinitis (EAU): induction of protective tolerance in primed animals. Clin Exp Immunol 109, 370-376.
126. Chen W et al (1998) Oral delivery of group A streptococcal cell walls augment circulating TGF-beta and suppress streptococcal call wall arthritis. J Immunol 161, 6297-6304.
127. Matzinger P (1994) Tolerance, danger and the extended family. Annu Rev Immunol 12, 991-1045.
128. Bullock D W et al (1975) Induction of T-lymphocyte responses to a small molecular weight antigen. II. specific tolerance induced in azebenzenearsonate (ABA)-specific T cells in Guniea pigs by administration of low doses of an ABA conjugate of chloroacetyl tyrosine in incomplete Freund's adjuvant. J Exp Med. 142, 261-74.
129. Leishman A J et al (1998) Immunological consequences of intervention in established immune responses by feeding protein antigens. Cell Immunol 183, 137-148.
130. Liu J Q et al (1998) Inhibition of experimental autoimmune encephalomyelitis in Lewis rats by nasal administration of encephlitogenic MBP peptides: synergistic effects of MBP-68-86 and 87-99, Int Immunol 10, 1139-1148.
131. Shi F D et al (1998) Nasal tolerance in experimental autoimmune myasthenia gravis (EAMG): induction of protective tolerance in primed animals. Clin Exp Immunol 111, 506-512.
132. Van Sechel A C et al (1999) Epstein-Barr virus-induced expression and HLA-DR-restricted presentation by human B cells of alpha B-crystallin, a candidate autoantigen in multiple sclerosis. J. Immunol. 162,129-135.
133. Van Stipdonk M J B et al (2000) Tolerance controls the encephalitogenicity of alpha B-crystallin in the Lewis rat. J. Neuroimmunol. 103, 103-111
134. Thoua N-M et al (2000) Encephalitogenicity and immunogenic potential of the stress protein alpha B-crystallin in Biozzi A B H (H-2g7) mice. J. Neuroimmunol. 104, 47-57.
135. Van Stipdonk M J B et al. (2000) T- and B-cell nonresponsiveness to self alpha B-crystallin prevents the induction of experimental autoimmune encephalomyelitis in SJL mice. Cell Immunol. In press
136. Van Noort, J. M., Bajramovic, J. J., Plomp, A. C. and Van Stipdonk, M. J. B. (2000) Mistaken self: a novel model that links microbial infections with myelin-directed autoimmunity in multiple sclerosis. J. Neuroimmunol. 105, 46-57.
137. Bajramovic, J. J., Van der Goes, A., Koevoets, C., Van Sechel A. C., Newcombe, J, Cuzner, M. L. and Van Noort, J. M. (2000) Presentation to T cells of alpha B-crystallin in MS lesions, an early event following inflammatory demyelination. J. Immunol. 164, 4359-4366.

1. A method of therapeutic treatment of a disease which is triggered or stimulated by Alpha B Crystallin, comprising administering a therapeutic agent comprising Alpha B crystallin or at least a specific immunogenic part thereof as a therapeutic agent to act against the disease.

2. The method of treatment according to claim 3, wherein the disease is an auto-immune disease.

3. The method according to claim 3, wherein the disease is multiple sclerosis.

4. The method of claim 3, wherein the treatment acts to induce tolerance against alpha B crystallin.
